(19) United States
(12) Patent Application Publication
(10) Pub. No.: US 2006/0229721 A1
(43) Pub. Date: Oct. 12, 2006

(54) Solid implant

(76) Inventor: David N. Ku, Atlanta, GA (US)

Correspondence Address: JONES DAY, 555 SOUTH FLOWER STREET FIFTIETH FLOOR, LOS ANGELES, CA, 90071, US

(21) Appl. No.: 11/450,772

(22) Filed: Jun. 8, 2006

Related U.S. Application Data
(63) Continuation-in-part of application No. 10/347,091, filed on Jan. 17, 2003.

(51) Int. Cl. A61F 2/12; A61F 2/02

(52) U.S. Cl. 623/8; 623/23.74

(57) ABSTRACT

An implantable prosthesis comprised of a solid elastomer made from a synthetic organic polymer that is biocompatible, compliant, and has water content greater than 5%. The solid construction prevents leaks and ruptures that are prevalent in other implants. The implant has the look, fit and feel of human tissue from various parts of the body.

### CROSS-REFERENCE TO RELATED APPLICATIONS

This is a continuation-in-part application of co-pending application Ser. No. 10/347,091, filed Jan. 17, 2003, which is incorporated by reference herein in its entirety.

### FIELD OF THE INVENTION

The present invention relates to the field of implantable prosthesis. In particular, the implants are used in a variety of plastic surgeries including but not limited to mastectomy, augmentation, or reconstruction.

### BACKGROUND

Over the years, many attempts have been made to come up with an appropriate implantable prosthesis for the breast that had the look and feel of natural breasts without any harmful side effects.

Early implants were made from foams such as polyethylene and cross-linked poly vinyl alcohol (“PVA”) that were hydrophobic and had little or no water content. Current commercial breast implants are silicone bags filled with either saline or silicone oil. The bag is a potential source of inflammation since the bag is made of silicone. Breast implants filled with silicone may elicit an immune response while implants filled with saline do not look or feel as natural. Further, all implants composed of an outer envelope and an inner filler material have the possibility for leaks, ruptures, or bleeding through the membrane of the envelope. Therefore, many types of fillers are being experimented with to try and create less harmful fillers as well as fillers that are less prone to leaks.

For example, U.S. Pat. No. 6,251,137 issued to Andrews et al., introduced an implantable prosthesis comprised of synthetic triglycerides.

U.S. Pat. No. 6,371,984 issued to Van Dyke et al., relates an implantable prosthesis filled with a keratin hydrogel.

U.S. Pat. No. 5,407,445 issued to Tautvydas, relates to a polyoxyethylene filler.

Thus, it is evident that a variety of filler materials and bags have been proposed and patented. However, a major shortcoming of all implants composed of an envelope and an inner filler, is that the envelope will inevitably leak, bleed, or even rupture in certain instances. The present invention proposes a different solution where the implant is completely solid throughout. Being solid throughout, the implant has no fluid that could leak, bleed or lead to a rupture. Even the problems with “gel-bleeds” have been solved. Gel-bleed is a term to describe the sticky residue that comes off an implant after it has been cut. Gel-bleeds have been associated primarily with silcone gels. However, the present invention does not “bleed” in any manner.

### SUMMARY OF THE INVENTION

This invention relates to a breast implant that is one solid material without a surrounding shell or bag. The device consists of a biocompatible elastomer of appropriate shape that has a modulus of elasticity that is less than 1 megaPascal.

Accordingly, it is an object of this invention to provide a solid one-piece implant that is not prone to ruptures or leaks. The use of a biocompatible elastomer of an appropriate size and shape will enable users to get breast augmentation or reconstructive surgery without fear of rejection by the body or damage from ruptures or leaks. The elastomer further has the look and feel consistent with normal breast tissue.

### BRIEF DESCRIPTION OF THE DRAWINGS

FIG. 1 is a perspective view of a breast implant.

FIG. 2 is a cross section view of a breast implant.

### DETAILED DESCRIPTION OF THE PREFERRED EMBODIMENT

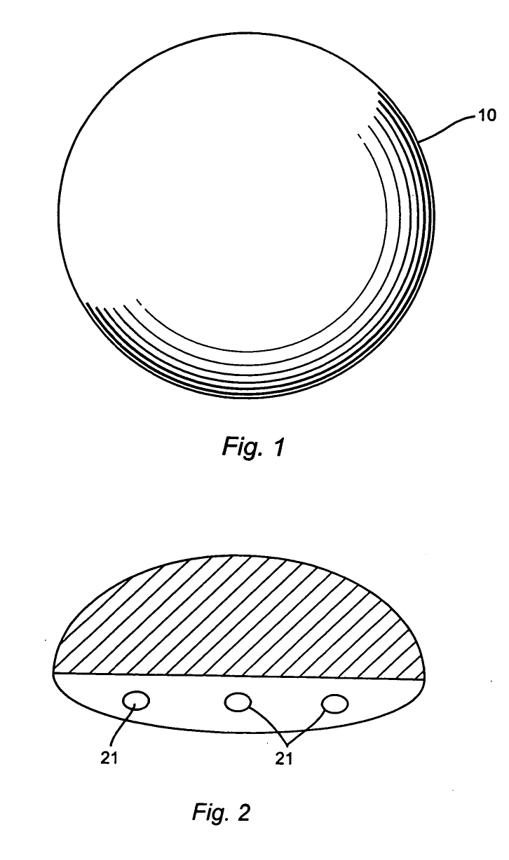

The present invention relates to a solid one-piece elastomer that is used as an implant 10. The implant 10 is suited for use in the breast but may be used in other parts of the human body. For example, addition uses contemplated are as a buttock, calf, male pectoralis or penile implant 10. FIG. 1 shows the implant 10 to be substantially circular in shape but the size and shape can vary depending upon the user's particular needs or preference. The implant 10 is made from a synthetic organic polymer that is biocompatible, compliant and has a water content greater than 5%. Biocompatibility prevents the implant 10 from being rejected by the human body. An inflammatory or immune response is typically generated when a foreign body is implanted into the human body. Molecules that are made from carbon and water are generally much more biocompatible than molecules containing silicon or other metals. The major problem with silicone has been that it is not an organic polymer, which may result in an immune response from the human body. The present invention utilizes an organic elastomer made from carbon atoms, not silicon atoms. Likewise, the body is predominantly composed of salt water. Water is clearly biocompatible. The property of hydrophyllicity (water loving) is a description of a material's affinity to water. Silicone and other implantable medical materials such as polyethylene and polytetrafluoroethylene are hydrophobic or water hating. If the implant 10 is formed from a material that contains water, then it must be hydrophilic and more likely to be biocompatible. Therefore, giving the implant 10 a water content of greater than 5% is beneficial for biocompatibility. Preferably, the solid implant 10 is made from a biocompatible elastomer with some water content such as hydrated polyurethane or polyvinyl alcohol.

In addition, the implant 10 is compliant. Compliance not only makes the implant 10 easier to work with but it gives the implant 10 a more natural look, fit and feel.

The implant 10 may be made by dissolving a polymer into saline to make a 10% weight solution. The solution is then poured into a mold in a controlled environment, preferably a globular shape, and preferably in a shape as shown in FIG. 1. The solution is then frozen to less than minus 5 degrees Celsius, preferably at a rate of less than 1 degree per minute. The implant 10 is then thawed to more than 2 degrees Celsius, preferably at a rate of less than 1 degree per minute. The freezing and thawing steps are repeated as needed to achieve solidity, preferably two times. Freezing and thawing at a rate of less than 1 degree Celsius facilitates an implant 10 having a tensile elongation length of greater than 400%. The implant 10 is then removed from the mold in a controlled environment and placed into a package with a water barrier seal.

FIG. 2 shows a cross section of an implant 10. The form is used only for illustrative purposes, the actual size and shape of the implant 10 may vary to suit the particular needs of the user. The cross section shows that the entire implant 10 is made from one solid piece of elastomer with no separate coverings or envelopes. The elastomer is made from one component and is homogeneous throughout. A single component is easier to manufacture and provides fewer points for inflammation. However, for a given implant 10, it may be desirable to provide several components to provide a bioactive reaction such as with a drug eluting or radioactive implant 10 to treat cancer. Thus, single and multiple component implants 10 are envisioned in this invention.

The implant 10 also has a compressive modulus of elasticity between 1 kiloPascal and 1 megaPascal. The solid implant 10 may have different areas with varying moduli of elasticity. The range in the modulus of elasticity allows the implant 10 to have variances that are consistent with normal breast tissue variations. For example, one part of the implant 10 may have a modulus of elasticity of 100 kiloPascals while another portion of the implant 10 may have a modulus of elasticity of 500 kiloPascals.

The implant 10 further has a tensile elongation length between 100% and 800%. In a preferred embodiment, the tensile length is greater than 400%, which gives the implant 10 similar “stretchiness” to normal breast tissue.

The implant 10 further has a smooth, textured, or modified surface, which aids in proper placement and fixation in the body. For example, a rough texture may cause increased adherence between the implant 10 and the surrounding tissues.

A tissue fixation component 21 may be combined with the solid implant 10 to enhance tissue fixation. A potential problem for a breast implant 10 made from a biocompatible material is that the implant 10 will migrate to a different anatomic location. Hence, it is useful to selectively encourage attachment at specific sites. The tissue fixation component 21 may be comprised of tabs or holes to allow the surgeon to suture the implant 10 to native body structures. Alternatively, the surface roughness and porosity may be tailored to allow for fibrotic in-growth and mechanical interlock. In another embodiment of the present invention, the material may include a biologically active agent that enhances attachment. In yet another embodiment of the present invention, a second material such as polyethylene may be molded in selective areas on the implant 10 to create fibrotic in-growth and mechanical interlock. For example, the tissue fixation component 21 may be in the form of a piece of Dacron® mesh that can be placed on the interior surface of the implant 10 to promote adhesion to the underlying chest wall or muscle fascia. Other methods may be used singly or in combination to achieve optimal attachment and these are anticipated.

The implant 10 is further designed to include a bioactive agent. A bioactive agent may be a synthetic drug or a naturally occurring molecule such as a hormone or growth factor. The implant 10 may contain such a bioactive agent to stimulate fibrotic attachment, reduce inflammation, retard cell proliferation, or many other bioactive properties depending on the agent. The invention contemplates the implant 10 that may contain the agent, not the agent itself. The bioactive agent allows for breast healing and local treatment.

The implant 10 is also given a uniform optical appearance for a variety of reasons. Since the implant 10 will be used in large part for cosmetic reasons, giving the implant 10 a pleasing look will aid in acceptance of its use. Additionally, the implant 10 is translucent which contributes to the aesthetics. Furthermore, the translucency aids in clinical examinations that look for breast lumps by shining a light from one side to the other to visualize dense lumps (trans-illumination). Breast tissue is primarily fat and has a translucency that glows under trans-illumination similar to the implant 10 described in the present invention. In contrast, other materials used for breast augmentations or reconstruction are opaque and stiff, rendering them visible under the skin.

The placing of the implant 10 in the chest wall makes a hydrophilic implant 10 preferable to hydrophobic ones such as polyethylene and cross-linked PVA that were hydrophobic and had little or no water. The hydrophilic implant 10 with a water content greater than 5% aids the implant 10 to have greater biocompatibility.

A further advantage of the present invention relates to mammograms. Mammograms enable doctors to take x-rays of the breast to check for tumors. Previous implants such as silicone implants posed problems by having a different density than the surrounding breast tissue effectively casting a shadow on the mammogram. Thus, it is very difficult to provide an accurate diagnosis using mammography with a patient that has saline or silicone breast implants. However, the present invention makes the implant 10 radiolucent due to its natural density that approximates normal breast tissue. Therefore, the present implant 10 does not hinder the use of mammograms.

The non-swelling nature of the implant 10 allows it to retain its shape and form. The implant 10 swells less than 10%.

In a further embodiment of the present invention, the implant 10 contains NaCl dissolved in the water content. The salt content can range between 0% and 2.0%. Preferably, the salt content is 0.9% by weight. Normal salt is Sodium (Na) and Chloride (Cl) which again aids in the biocompatible nature of the implant 10.

In yet a further embodiment of the present invention, the implant 10 may be designed to hold a cancer therapeutic agent for local treatment of cancer. The implant 10 may be designed with a chamber to hold a chemotherapeutic agent or radiological seed for brachytherapy. This chamber may be an actual void that is filled at another time or a volume that contains the agent within the solid material of the implant 10.

It is readily apparent to those skilled in the art that numerous modifications, alterations, and changes can be made without departing from the inventive concept described herein. The invention, therefore, is not to be restricted except in the spirit of the appended claims.

1. A space-filling breast implant comprising a solid one piece hydrated polymer, wherein the polymer is biocompatible, compliant, and has a water content greater than 5% and a tensile elongation length greater than 400%.

2. The implant of claim 1, wherein the polymer is selected from the group consisting of hydrophilic polyvinyl alcohol, polyurethane and polyethylene, and the polymer has a compressive modulus of elasticity between 1 kiloPascal and 1 megaPascal.

3. (canceled)

4. (canceled)

5. The implant of claim 1, wherein the polymer is a hydrated polyvinyl alcohol and has a smooth surface.

6. (canceled)

7. The implant of claim 1, wherein the polymer is a hydrated polyvinyl alcohol and comprises a tissue fixation component.

8. The implant of claim 7, wherein the hydrated polyvinyl alcohol and comprises a bioactive agent.

9. The implant of claim 7, wherein the hydrated polyvinyl alcohol is optically translucent.

10. The implant of claim 7, wherein the hydrated polyvinyl alcohol is radiolucent.

11. The implant of claim 1, wherein the polymer is a hydrated polyvinyl alcohol and has portions with different moduli of elasticity.

12. The implant of claim 1, wherein the polymer is a hydrated polyvinyl alcohol and is homogenous.

13. The implant of claim 1, wherein the hydrated polymer swells less than 10% when placed in a body.

14. (canceled)

15. The implant of claim 1, wherein the polymer is a hydrated polyvinyl alcohol implant and contains dissolved sodium chloride salt.

16. (canceled)

17. (canceled)

18. (canceled)

19. A breast implant comprising a solid space-filling implant for breast reconstruction formed from a synthetic, organic and hydrated polymer, selected from the group comprising, polyvinyl alcohol, polyurethane and polyethylene wherein said implant is biocompatible, has a modulus of elasticity between 100 kpascals and 500 kPascals, a tensile elongation length greater than 400%, has a water content between 85% and 95% by volume, has NaCl salt content of 0.9% by weight, has porosity to allow for mechanical fixation by fibrous tissue, and being solid, cannot leak from manual manipulation or cuts to the surface at any point.

20. A breast implant comprising a solid space-filling implant for plastic surgery formed from a synthetic, organic, and hydrated polymer, selected from the group comprising polyvinyl alcohol, polyurethane and polyethylene, wherein said implant is biocompatible, compliant, has a water content greater than 5%, has a compressive modulus of elasticity between 100 kpascals and 500 kpascals, a tensile elongation length greater than 400%, has NaCl salt content of 0.9% by weight, has a tissue fixation component, has a bioactive agent within the solid implant material, has more than one modulus of elasticity, is optically translucent, and being solid, cannot leak from manual manipulation or cuts to the surface at any point.

21. A buttock prosthesis comprising a solid space-filling implant for buttock enhancement formed from a synthetic, organic and hydrated polymer, wherein said prosthesis is biocompatible, has a modulus of elasticity between 100 kpascals and 500 kpascals, a tensile elongation length greater than 400%, has a water content between 85% and 95% by volume, has NaCl salt content of 0.9% by weight, has a porosity to allow for mechanical fixation by fibrous tissue, and being solid, cannot leak from manual manipulation or cuts to the surface at any point.

22. A calf implant comprising a solid space-filling implant for calf muscle shaping formed from a synthetic, organic and hydrophilic hydrated polymer, wherein said implant is biocompatible, has a modulus of elasticity between 100 kpascals and 500 kPascals, a tensile elongation length greater than 400%, has a water content between 85% and 95% by volume, has NaCl salt content of 0.9% by weight, has a porosity to allow for mechanical fixation by fibrous tissue, and being solid, cannot leak from manual manipulation or cuts to the surface at any point.
